(19) United States
(12) Patent Application Publication
(10) Pub. No.: US 2005/0214311 A1
(43) Pub. Date: Sep. 29, 2005

(54) Novel complexes for inducing an immune response

(76) Inventors: Gavin Robert Screaton, Oxfordshire (GB); Katharina Anna Simon, Oxfordshire (GB); Awen Myfanwy Gallimore, South Wales (GB)

Correspondence Address: Pearl Cohen Zedek Latzer, Suite 1001, 10 Rockefeller Plaza, New York, NY, 10020, US

(21) Appl. No.: 10/505,420

(22) PCT Filed: Feb. 25, 2003
(86) PCT No.: PCT/GB03/00811

§ 371 Date: May 9, 2005

(30) Foreign Application Priority Data
Feb. 25, 2002 (GB) 0204348.7
Oct. 15, 2002 (GB) 0223947.3

(51) Int. Cl. A61K 39/00; A61K 39/12; A61K 39/02

(52) U.S. Cl. 424/185.1; 424/186.1; 424/190.1

(57) ABSTRACT

The invention relates to induction of immune responses against target antigens using Fas ligand as an adjuvant. Melanoma cell lines transfected with FasL are shown to be rejected more efficiently than untransfected cells, and rejection is shown to be antibody-mediated. The invention provides immune complexes comprising target antigens and Fas ligand, pharmaceutical compositions comprising such immune complexes, and methods for generating immune responses, as well as methods for identifying cell-specific antigens, particularly tumour specific antigens, using cells expressing FasL.

The present invention relates to novel complexes for inducing an immune response in an. individual. Particularly, but not exclusively, the invention relates to methods of inducing an immune response against one or more antigens using Fas ligand as an adjuvant.

Immune surveillance by both the innate and acquired arms of the immune system plays a part in eliminating some transformed cells. However the protection afforded is less than previously thought¹. The frequent appearance of tumours in humans illustrates the fact that many tumour cells are ignored, develop mechanisms to escape, or at least tip the balance of an immune response preventing their elimination. Fas Ligand (FasL), a member of the TNF family of proteins, is well characterised for its role in triggering apoptosis. Expression of Fas ligand by activated lymphocytes allows them to kill target cells expressing Fas². However, expression of FasL by these cells is a two edged sword as activated lymphocytes themselves express Fas and become susceptible to death. The Fas/FasL axis thus limits an immune response and is a major player in the process of activation induced cell death³.

FasL expression is normally tightly restricted to activated lymphocytes. However, it has been demonstrated that some non-lymphoid tissues such as the eye and testis can express FasL⁴⁻⁶ This has been proposed to underlie the immune privilege enjoyed by these organs, in effect allowing them to kill infiltrating lymphocytes i.e. “kill the killers”. Shortly after these initial reports some tumours were demonstrated to express FasL and this was proposed to be a mechanism used to escape an immune response^{7, 8}. These findings are now the subject of much debate as forced expression of FasL on tumours or transgenic expression in organs such as the pancreas often leads to a brisk neutrophil infiltration and elimination of tumour or damage to the transgenic organ⁹.

In this study the inventors show that the molecule Fas ligand, behaves as an adjuvant to generate an anti-tumour response. For the first time, the inventors show that the immunity is mediated by tumour-specific antibodies, which can recognise and lyse the tumour cell line. The determination that this is an antibody based immunity suggests the potential for a long term tumour immunity. Following this work, the inventors have appreciated that this system when used to generate tumour-specific antibodies may help in the definition of new tumour antigens, and be valuable for the design of better vaccines and therapies of tumours, e.g. melanoma.

Specifically, the inventors have used the B16F10 melanoma model to study the effects of FasL expression. This tumour is poorly immunogenic and thus a good model to test strategies to enhance tumour immunity. They found that mice reject tumour cells transfected with FasL and go on to develop tumour specific immunity. Unlike previous studies where immunity was shown to be mediated by CD8 lymphocytes, this immunity can be transferred by serum. Here the inventors show that immune responses directed to melanocyte differentiation antigens, are indeed induced by FasL expressing tumour cells.

At its most general, the present invention provides materials and methods for inducing an antibody immune response in an individual against an antigen, e.g. a tumour associated antigen, bacterial antigen, viral antigen etc, using FasL as an adjuvant. In a preferred embodiment, the antigen is a tumour associated antigen and therefore, the following text concentrates on tumour associated antigen. However, it will be apparent to the skilled person that the following aspects of the invention may also by applicable to other antigens, e.g. those derived from bacteria or viruses.

The inventors have determined that FasL can be used to elicit a T-cell response, particularly a CD4 immune response. Further, the inventors have shown in detail that FasL has the ability to activate/mature dendritic cells to enhance immune responses.

Further, the present invention provides screening methods for identifying specific tumour associated antigens and methods for producing specific monoclonal antibodies for use in the treatment of cancer.

Thus, in a first aspect, there is provided an immunocomplex comprising a tumour associated antigen and FasL. The tumour associated antigen and FasL may be in the form of nucleic acid, which may be translated in an individual to produce a fusion protein comprising both the tumour associated antigen and the FasL polypeptides. Alternatively a nucleic acid encoding one of the antigen and FasL may be coupled to a nucleic acid encoding the other. Alternatively, the immunocomplex may comprise the tumour associated antigen and the FasL as a fusion protein. In a preferred embodiment, the tumour associated antigen is provided by transfecting a tumour cell with FasL such that the ligand is expressed by the transfected cell along with the tumour associated antigens.

The inventors have illustrated this aspect of the invention by providing a melanoma cell transfected with FasL. However, as set out above it will readily be appreciated that similar immunocomplexes may be used to immunise an individual against any suitable antigen from a source other than a tumour, such as an antigen from a pathogen, e.g. a bacterial or viral antigen. The antigen may be an isolated molecule (preferably a protein molecule) purified from a pathogen, such as a bacterium or virus, or expressed in recombinant form. Alternatively, the antigen may be a live, attenuated or killed pathogen. FasL may be administered with the pathogen as free protein, or may be associated with the pathogen either non-covalently or covalently, e.g. by chemical coupling. Alternatively the pathogen may express or contain FasL as a result of genetic engineering (see below).

The invention further provides a pharmaceutical composition comprising said immunocomplex according to the first aspect of the invention.

In a preferred embodiment, the immunocomplex further comprises an immune adjuvant which is capable of producing a synergistic effect with FasL. Such an immune adjuvant may be anti-CD25 monoclonal antibodies, which are able to deplete regulatory/suppressor T-cells.

The invention further provides a method of inducing an immune response in an individual against an antigen, comprising the step of administering an immunocomplex, a nucleic acid, or a pharmaceutical composition according to the first aspect of the invention. An immune adjuvant as described above may also be administered to enhance the immune response so induced.

In a second aspect of the present invention, there is provided a method of inducing an immune response in an individual against a tumour cell, as either a therapeutic or prophylactic treatment against cancer, said method comprising the steps of
(a) obtaining a tumour cell e.g. from said individual;
(b) transfecting said cell with FasL such that said cell expresses FasL; and
(c) administering said transfected tumour cell to said individual so as to induce an antibody immune response against said tumour.

The method may further comprise the administration of an immune adjuvant to synergise with the FasL effect, e.g. anti-CD25 monoclonal antibodies which are capable of depleting CD25 expressing cells.

The immune adjuvant may be administered before, during or after the administration of the transfected tumour cell.

An alternative embodiment of the second aspect would be to obtain a tumour cell from a source other than the individual to be treated, such as an established cell line.

Of course, the second aspect of the present invention can also be adapted to induce an immune response in an individual against an antigen from a pathogen, such as a bacterial or viral antigen. In a preferred embodiment, the pathogen may be engineered to express FasL. Thus a pathogen cell, such as a bacterium, may be transfected or transformed with a nucleic construct enabling expression of FasL, e.g. to be displayed at the surface of the cell or to be secreted by it. Likewise, a virus may be engineered so that FasL is incorporated into the virus particle. This may be achieved by engineering of the viral genome so that the protein is expressed, for example, as a fusion with a virus protein. Alternatively the FasL protein may be expressed in a virus-producing cell so it is incorporated into the virion particle, e.g. in a membrane budded from the producer cell's plasma membrane. Pathogens, including viruses, which contain, display or express FasL as a result of engineering of the pathogen or a producer cell may themselves be considered immunocomplexes of the first aspect of the invention.

The invention therefore further provides a method of inducing an immune response in an individual against a pathogen, as either a therapeutic or prophylactic treatment against infection with said pathogen, said method comprising the steps of engineering said pathogen to express FasL, and administering said engineered pathogen to said individual so as to induce an antibody immune response against said pathogen.

Also provided is the use of a pathogen engineered to express FasL in the preparation of a medicament for the therapeutic or prophylactic treatment against infection with said pathogen.

In a third aspect of the invention, there is provided a screening method for identifying tumour-specific antibodies, said method comprising the steps of
(a) transfecting a tumour cell with FasL;
(b) vaccinating a test animal with said transfected tumour cell;
(c) collecting serum from said test animal;
(d) identifying antibodies specific for said tumour cell from the serum.

The method may further comprise the step of producing a pharmaceutical composition comprising said identified antibody. The antibody may be polyclonal or monoclonal. Preferably, the method will further comprise the step of producing monoclonal antibodies using the identified antibody in standard methods.

In a preferred embodiment of the present invention, there is provided a method for identifying specific tumour associated antigens using antibodies raised against an immunocomplex according to the first aspect or the antibodies isolated by a method according the third aspect. The method may comprise the steps of contacting/screening said antibody with a plurality of potential tumour associated antigens obtained from the tumour cell used to vaccinate the test animal. The potential tumour associated antigens may conveniently be displayed using an expression library (e.g. a phage expression library) or on a solid support e.g. a 2d SDS PAGE followed by a western blot. The antibody may then be contacted/screened with the potential antigens and the specific binding between the antibody and the antigen identified by labelling or other routine techniques. The antigen may then be characterised using standard methodology known to the skilled person.

It will be apparent to the skilled person that the methods of the third aspect of the invention may be extended to identification of antigens in any cell type, and of the generation of antibodies against such antigens.

Aspects and embodiments of the present invention will now be illustrated, by way of example, with reference to the accompanying figures. Further aspects and embodiments will be apparent to those skilled in the art. All documents mentioned in the text are incorporated herein be reference.

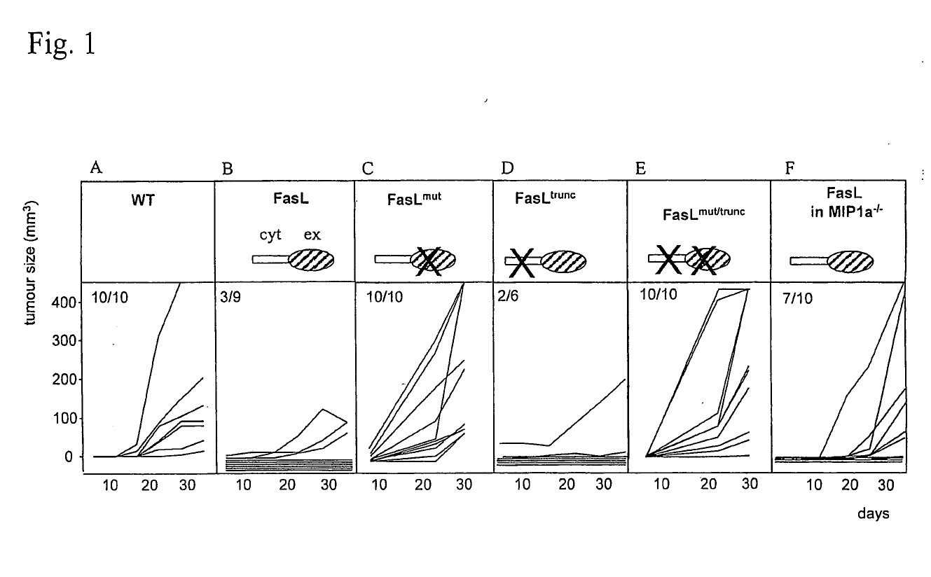

FIG. 1

Rejection of FasL Expressing Tumours.

C57BL/6 mice were injected sc with 5×10⁵ tumour cells. Cells were either untransfected B16WT (A) or stably transfected with full length FasL (B), full length FasL with a mutation in the Fas binding site (FasL^{mut} C), truncated FasL (FasL^{trunc} D) or truncated FasL with no binding to Fas (FasL^{mut/trunc} E). Mip1α deficient mice were given B16F10 transfected with full length FasL (F). Tumour growth was measured twice a week over a period of 5 weeks. Numbers represent tumour bearing mice/total.

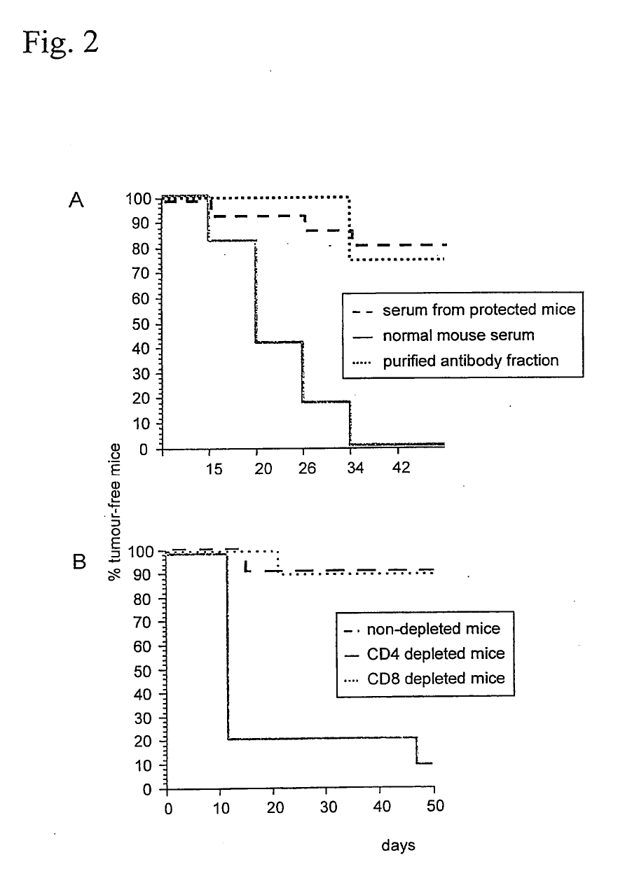

FIG. 2

Serum from Protected Mice Transfers Tumour Immunity and Protection is Dependent on CD4 T cells.

200 μl of serum or 1.2 mg purified antibodies from protected mice was transferred into naïve C57B/L6 mice and mice were challenged the following day with 5×10⁵ or 2×10⁵ B16WT respectively (A). C57BL/6 mice were depleted of CD4 or CD8 T lymphocytes, injected with 10×10⁶ B16FasL and then challenged with 5×10⁵ B16F10 (B).

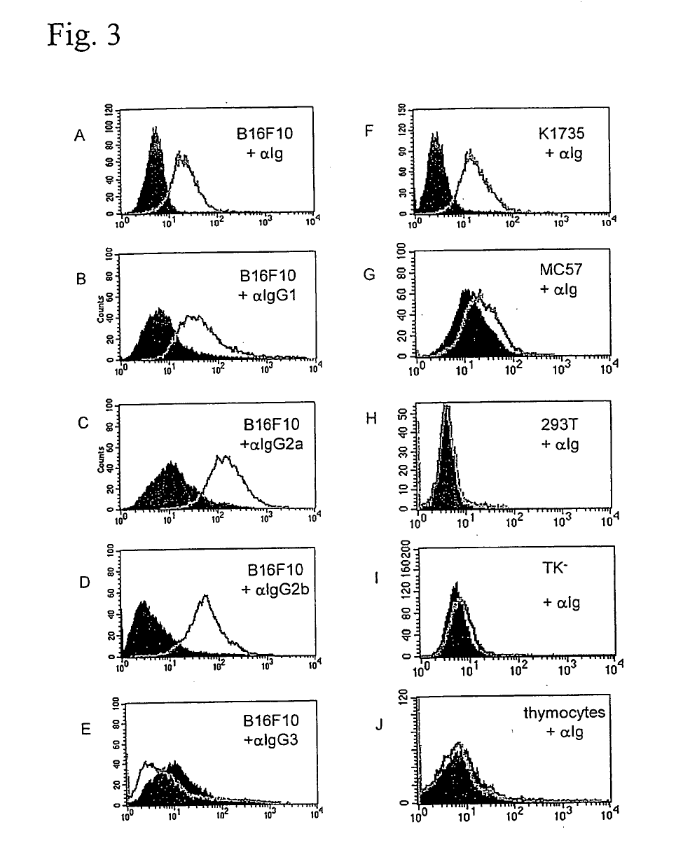

FIG. 3

Serum from Protected Mice Surface Stains B16F10 and Includes the Isotypes IgG1, IgG2a, IgG2b.

Tumour and primary cells were stained by indirect immunofluorescence using the indicated secondary antibodies and analysed by FACS. Dark shading-control serum, open line-serum from protected mice.

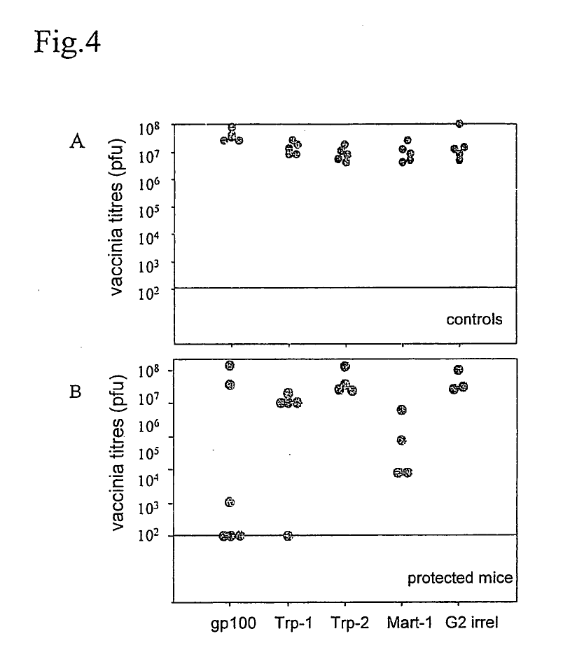

FIG. 4

Protected Mice Show Responses to Melanocyte Antigens.

Mice were injected with vaccinia virus expressing the melanocyte differentiation antigens gp100, Trp-1, Trp-2, Mart-1 or an irrelevant antigen G2. Five days later viral titers were determined in ovaries. Naive mice (A) protected mice (B). This experiment was repeated twice with similar results. The dashed line represents the limit of sensitivity of the assay.

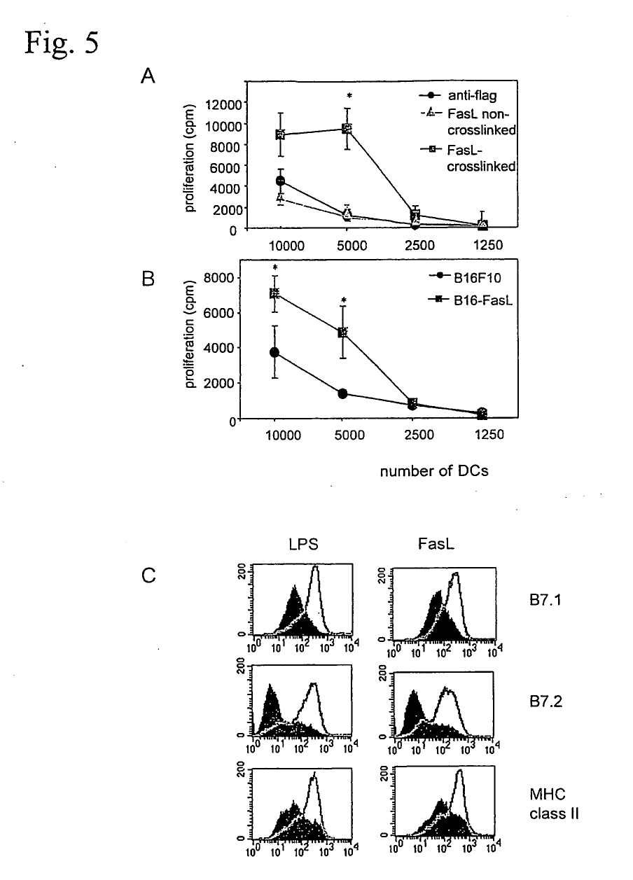

FIG. 5

Dendritic Cells Mature in the Presence of B16FasL.

After culture in GM-CSF and IL-4 bone marrow dendritic cells from C57BL/6 mice were matured overnight with anti-FLAG mAb as control, FasL non-crosslinked or crosslinked with anti-FLAG (A), irradiated B16WT or B16FasL (B). BALB/c spleen cells were added to increasing numbers of dendritic cells and proliferation was measured by ³H thymidine incorporation. Background proliferation in the absence of spleen cells was subtracted. This experiment was repeated three times with similar results. *P<0.05 Student's T test. Maturation markers of dendritic cells were stained by mAb after maturation with recombinant FasL overnight. Dark shading-anti-FLAG alone, open line-maturation with LPS or recombinant FasL crosslinked (C).

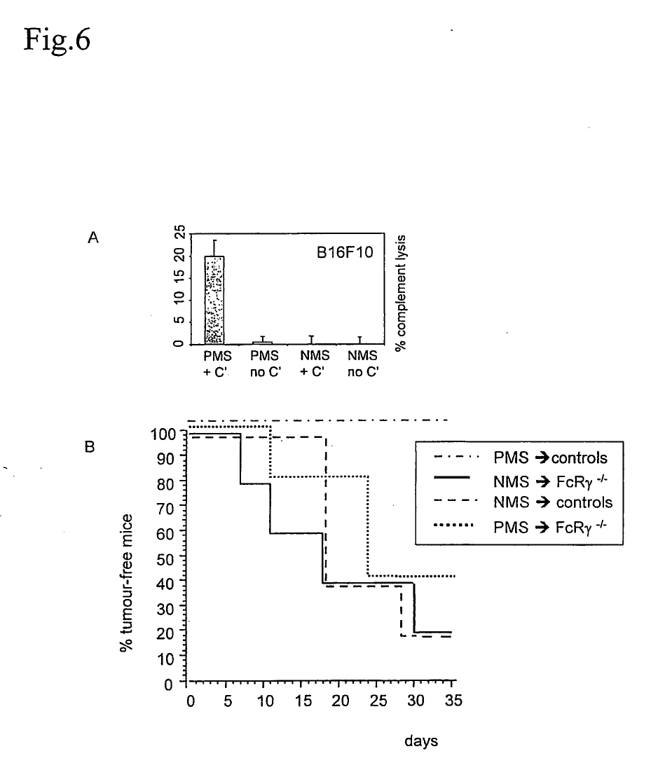

FIG. 6

The Serum Mediated Tumour Immunity is FcR Dependent

(A) Serum from protected mice (PMS) was used in a complement lysis assay (A). Normal mouse serum (NMS) and wells without complement were included as controls. This panel is representative of three separate experiments. (B) NMS or PMS was injected in FcRγ deficient mice or age matched C57BL/6 controls, then challenged with 1×105 B16WT.

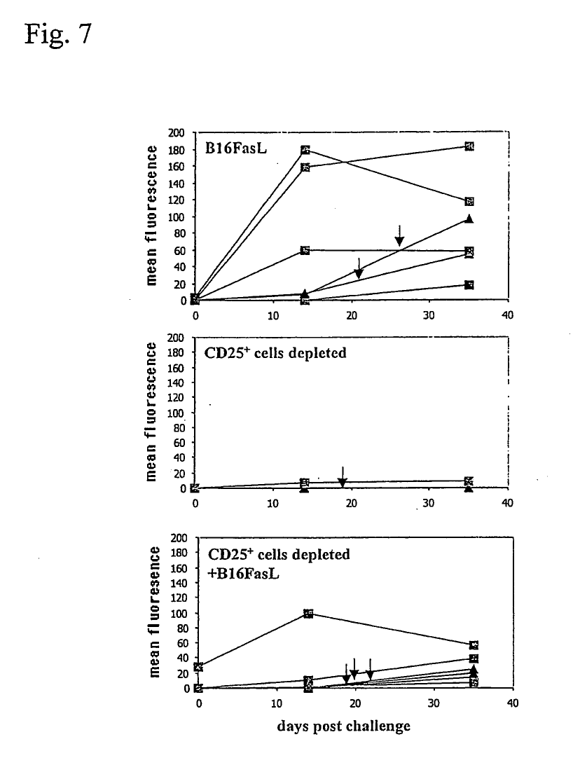

FIG. 7

Development of High Serum Titers Coincides with Tumour Growth in Individual Mice

Mice were treated either with live B16FasL or CD25+ regulatory cell depleting antibody or both. 4 weeks later, mice were challenged with 0.5×105 B16WT. At regular intervals serum was taken and analysed for staining of B16F10 and mean fluorescence represented here. Lines indicated with squares show tumour-free mice and those shown with triangles and arrows show mice that developed tumours. Most mice that develop tumours have low antibody titers or developed antibodies too late to control tumour growth.

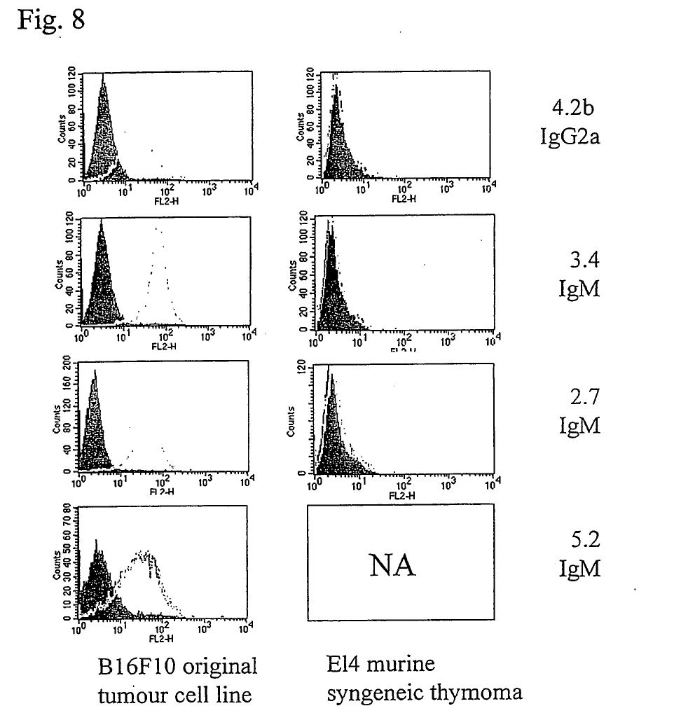

FIG. 8

Generation of Monoclonal Antibodies from Tumour-Immune Mice, Isotypes and Specificity

Monoclonal antibodies specific for the original tumour cell line do not stain non transformed syngeneic cells (or transformed syngeneic non-melanoma cell lines). Monoclonal antibodies were raised from the spleen of tumour-immune mice by fusion with NS-1 fusion partner. Screening was performed by FACS analysis of B16F10, the original tumour cell line. Shaded histograms show staining with irrelevant isotype control.

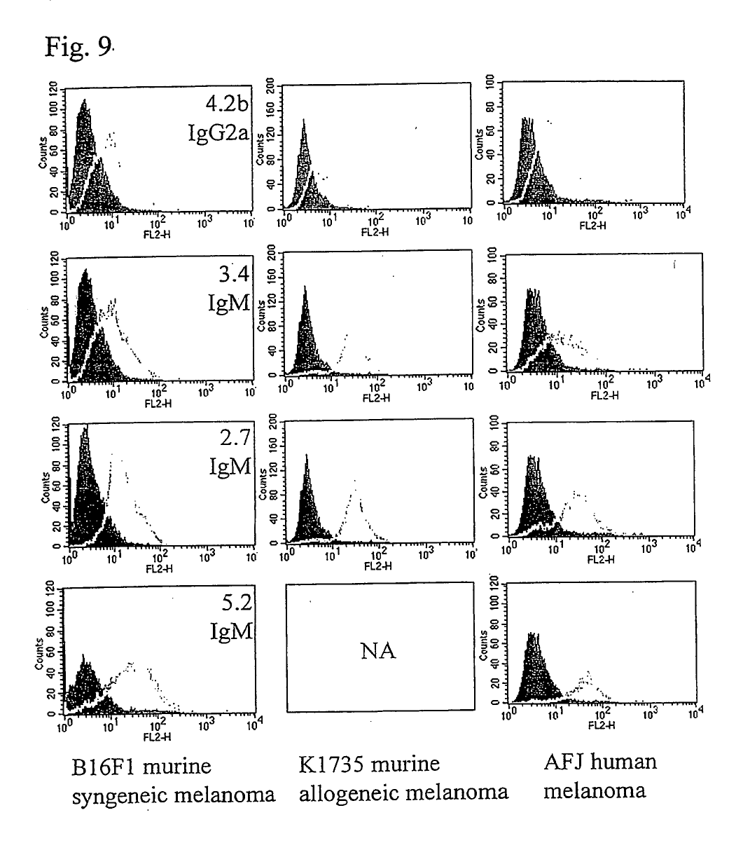

FIG. 9

FACS Analysis of Melanoma Cell Lines with Monoclonal Antibodies

All four monoclonal antibodies are specific for melanoma antigens. FACS staining was positive for a syngeneic melanoma, an allogeneic melanoma, and a human melanoma cell line.

### FAS BINDING AND NOT THE CYTOPLASMIC CHAIN OF FAS MEDIATE TUMOUR REJECTION OF FasL EXPRESSING TUMOURS

Counter to initial observations a number of investigators have now demonstrated that FasL transfected tumour cells are more efficiently rejected than FasL negative cell lines upon their injection in vivo. The brisk tumour rejection seems to be mediated in the main by infiltrating neutrophils. Although it seems clear that once present, neutrophils can kill the tumour cells, little is known about how the neutrophils are attracted in the first place. In a first series of experiments the inventors established a model of FasL mediated tumour rejection using the well-characterised murine melanoma cell line B16F10. Stable cell lines expressing a FasL were established (B16FasL) and injected subcutaneously (sc) into C57BL/6 mice.

Examination of the site of injection revealed a small but clearly palpable swelling at the injected site up to 2 weeks after injection. All 10 mice injected with the parental or “wild type” B16F10 (B16WT) developed tumours (FIG. 1a), whereas 6 of 9 rejected the FasL expressing tumour (FIG. 1b) confirming previous studies using the B16 model and other murine tumour models⁸. The inventors next confirmed that this was due to a direct interaction between Fas and FasL by expressing a mutant form of FasL FasL^{mut}), which contains a single point mutation in the extracellular domain, that prevents Fas binding (Y218R) 10. This mutation abolished rejection and tumours developed in all 10 mice (FIG. 1c). This is consistent with previous observations that FasL can activate neutrophils via Fas and that tumour rejection is prevented in lpr mice that express a mutated non-signalling Fas^{11, 12}. FasL has a long and conserved cytoplasmic domain rich in proline residues^{13, 14}, which could potentially allow “reverse signalling” via FasL to the tumour cell, perhaps causing the secretion of neutrophil chemoattractants. However deletion of this domain (FasL^{trunk} FIG. 1d, 1e)has no influence, implying that it is the engagement of Fas rather than FasL reverse signalling, that leads to tumour rejection.

Further support for role of neutrophils was obtained when tumours were injected into Mip1α deficient mice. Mip1α has previously been shown by others to act as a neutrophil chemoattractant^{15, 16}. In agreement with these observations, the ability to reject B16FasL was impaired in Mip1α deficient mice. Although the kinetics of tumour growth were slightly slower for the B16FasL tumour versus the B16WT control^{7/10} mice injected with the B16FasL developed tumours suggesting a possible role for this neutrophil chemoattractant in FasL mediated tumour rejection (FIG. 1f).

FasL Expressing Melanoma Induces Tumour Immunity

Of 123 mice vaccinated with a large dose of live B16FasL, 56% remained tumour-free (Table 1). When tumour-free vaccinated mice were challenged with 5×10⁵ B16WT about half of the 69 mice (54%) rejected the second tumour. These results were confirmed using two independent sublines of B16WT. For subsequent experiments it was established that 10×10⁶ irradiated B16FasL could protect against a second tumour. This has the advantage of preventing the growth of the FasL transfected tumours occurring in approximately 44% of mice given live B16FasL. All of the mice given irradiated B16FasL remained tumour free. 70% of these mice treated with irradiated B16FasL developed tumour immunity (n=90) and rejected tumour when challenged with B16WT. As expected, irradiated B16WT did not give rise to tumour immunity (Table 1), confirming results of other studies¹⁷. Similar results were obtained using several different clones expressing FasL (data not shown).

Tumour Immunity can be Transferred by Serum, but not by CD4⁺ or CD8⁺ T Cells.

Mice were vaccinated with B16 FasL and challenged with B16WT tumour. Animals that rejected the tumour challenge were then selected for further study,and are subsequently referred to here as “protected mice”. To determine which cells are critical for tumour immunity, protected mice were depleted of CD4⁺ or CD8⁺ T cells by administration of depleting antibodies in vivo. Mice were then re-challenged with B16WT. Both CD4⁺ and CD8⁺ T cells depleted mice were still able to reject the tumour (Table 2). Similarly the transfer of purified CD8⁺ or CD8⁺/CD4⁺ lymphocytes from protected mice did not prevent tumour growth. The inventors also tested fresh ex vivo killing of B16WT or targets infected with vaccinia viruses encoding melanocyte differentiation antigens. At a variety of effector target ratios they detected no lytic activity from splenocytes or purified CD8⁺ lymphocytes (data not shown).

The lack of effect using depleting antibodies was surprising, as it has previously been shown that FasL can prime CD8 T cell responses^{18, 19}. This prompted the inventors to test for the development of a humoral anti-tumour response. Serum was collected from protected mice leaving a minimum period of 7 weeks after injection of B16FasL. This was pooled and then injected into naïve mice, which were subsequently challenged with 5×10⁵ B16WT. 14/17 mice given serum were protected from challenge, whereas all of 17 given normal mouse serum developed tumour (FIG. 2). Serum was still protective when obtained 6 months after vaccination and serum from unvaccinated tumour-bearing mice failed to protect (data not shown).

To show that the tumour protection was mediated by antibody the immunoglobulin fraction of the serum from B16FasL treated or control mice was purified over a Protein L column, known to bind a high proportion of murine IgC and IgM. 1.2 mg of purified Ig was injected intravenously followed by challenge with 2×10⁵ B16WT. In this experiment all four mice given control Ig developed tumour at day 26 whilst 3/4 of mice given serum from protected mice remained tumour free (FIG. 2A).

To determine whether tumour immunity is established in a T cell dependent manner, mice had been depleted of CD4 or CD8 T cells before vaccination with B16FasL. Depletion of CD8 did not affect the ability of protected mice to reject a secondary challenge with B16WT. However, the majority of protected mice depleted of CD4 developed tumour when challenged (FIG. 2B). This was confirmed in a second experiment where mice were thymectomised and treated with depleting antibodies for CD4 and CD8 T cells. These profoundly T cell deficient mice were injected with B16FasL and subsequently challenged with B16WT. Ten control, non-depleted, mice rejected the tumour, whilst 3/10 T cell deficient mice rejected tumour. Thus, although mediated by antibody, the immune response to B16WT requires CD4 T cell help.

The Serum from Immunised Mice Reacts with Melanoma Antigens and can Trigger Complement-Mediated Lysis of B16F10.

B16WT (melanoma line from C57BL/6 mouse strain), K1735 (melanoma line from the C3H mouse strain), MC57 (methylcholanthrene-induced fibrosarcoma cell line from C57BL/6 mice), 293T cells (human embryonal kidney

fibroblast), HuTK-143B (TK⁻⁾ cells (human osteoblastoma cell line) and thymocytes from C57BL/6 mice were stained by indirect immunofluorescence using serum from protected, tumour bearing or control mice. FACS analysis showed no staining with either control serum or serum from tumour bearing mice, whereas strong staining of B16WT was seen using the serum from protected mice (FIG. 3a). Serum also stained the other melanoma cell line K1735 (FIG. 3f) there was a small amount of staining of MC57 cells but not 293T, TK⁻ or thymocytes from C57BL/6 (FIG. 3G-J). All cells were cultured under the same conditions and the negative results on some of these result indicate that antigens recognised by the polyclonal serum are not due to contaminants present in the culture medium. Seven weeks after injection of B16 FasL antibodies were of the IgG1, IgG2a and IgG2b isotypes (FIG. 3B-D). No specific antibodies of the IgG3 (FIG. 3E) or IgE or IgM isotypes were detected in the serum of protected mice, although specific IgM antibodies were present in the earlier phase of the immune response (less than seven weeks after injection of B16FasL).

Tumour Immune Mice React to Melanocyte Differentiation Antigens

In a search for antigen specific responses induced by vaccination with B16FasL the inventors took advantage of a panel of vaccinia viruses expressing the melanocyte differentiation antigens gp100, Trp-1, Trp-2, Melan-A/Mart-1 or an irrelevant antigen G2, a glycoprotein from lymphocytic choriomenigitis virus^{20, 21}. When injected into naïve mice these viruses replicate and this can be assayed by counting plaque-forming units from ovaries infected with vaccinia virus. If the mouse has pre-existing immunity against melanocyte antigens carried by the virus then replication will be inhibited and the ovary plaque count reduced. Vaccinia titres measured in ovaries were consistently high in control mice (FIG. 4a). The majority of vaccinated mice infected with vac-gp100 cleared the virus or had reduced viral load (4 out of 6, FIG. 4b), one vaccinated mouse cleared vac-Trp-1 and viral titres to Mart-1/MelanA were lowered in 2 mice by a factor of 10³ as compared to controls (FIG. 4b). It is possible that B16FasL will also break tolerance to other tumour associated and tumour specific antigens expressed by B16 and the protection the inventors observe may well be improved by such a polyspecific response. The observation that approximately 20% of mice exhibited depigmentation at the site of tumour inoculation (B16FasL) further supports the findings that immune responses to melanocyte antigens were induced in these mice.

B16FasL can Mature Dendritic Cells.

To gain more insight into how FasL expression might help prime tumour specific responses, the inventors examined whether B16FasL could mature dendritic cells (DC). Bone marrow derived DC's were cultured in vitro for 4 days, matured in the presence of irradiated B16WT, B16FasL, or soluble FasL-FLAG fusion protein. FasL was incubated either alone or crosslinked with anti-FLAG mAb, then washed and added to allogeneic splenocytes in a 4 day proliferation assay using tritiated thymidine. Activation of splenocytes was improved significantly when DC's were matured in the presence of B16FasL or soluble crosslinked FasL (FIG. 5A+B). The maturation markers MHC class II, CD83 and CD86 were also upregulated on DC's upon culture with FasL (FIG. 5C).

Antibody-Mediated Tumour Immunity is Dependent on Fc Receptors (FcRs)

As complement and Fc receptor mediate effector functions of antibodies the inventors sought to determine the role of

these two components during the tumour rejection in the protected mice. In an in vitro complement lysis assay protected but not preimmune serum gave significant lysis of B16WT (FIG. 6A). Next they depleted complement in protected mice by repeated injections (30 μg/mouse each injection) of Cobra venom factor (CVF) which depletes C3 (Shapiro et al., 2002). The mice were then challenged with B16WT. In two separate experiments, 4/5 controls and 7/8 CVF treated mice rejected tumour.

To look for a role of antibody dependent cell mediated cytotoxicity (ADCC) the inventors injected protected serum into Fc receptor gamma chain deficient mice (FcRγ^{31 /-} which lack: FcγRI, FcγRIII and FcεRI) (Takai et al., 1994). Mice were given serum from protected or control mice and then challenged with B16WT, All control mice were protected from tumour when injected with protected serum, whereas in the FcRγ^{31/-} the same serum no longer provided protection (FIG. 6B).

FasL Immunocomplex in Conjunction with Anti-CD25 Antibodies

Using a murine model, the inventors have further found that induction of immune responses capable of controlling growth of the melanoma cell-line B16, can be achieved in different ways. Firstly, removal of CD25⁺ regulatory cells using CD25-specific monoclonal antibodies in vivo results in tumour rejection following inoculation of mice with live B16 cells (approx. 50% of mice). In this case, immunity is mediated by CD4⁺ T cells. Secondly, the inventors have found that tumour immunity can be generated by inoculating mice with B16 cells engineered to express FasL in accordance with the present invention(approx 60% of mice). In this case, immunity is mediated by antibodies. Several independent experiments have, however, shown that long-term immunity can be achieved in nearly 100% of mice immunized with B16FasL in the absence of CD25⁺ regulatory cells (i.e. when B16 FasL transfected cells are given together with depleting anti-CD25 monoclonal antibodies, followed by challenge with the parental B16 cells).

The superior protection against tumour growth obtained using the latter protocol could reflect induction of both an antibody response and a CD4⁺ T cell response capable of rejecting B16. Alternatively, depletion of CD25³⁰ regulatory cells may enhance the antibody response induced by B16FasL.

A kinetic study of individual mice shows firstly that the development of serum antibodies coincides with tumour growth, and also that mice which develop tumours either have low antibody titers or develop antibody too late to control

tumour growth (FIG. 7).

Generation of Monoclonal Antibodies from Tumour-Immune Mice

Monoclonal antibodies were raised from the spleen of tumour-immune mice. FIGS. 8 and 9 show characterisation of four of these antibodies, which were specific for melanoma antigens and did not stain a syngeneic thymoma cell line.

Discussion

The immune system has evolved to recognise and kill foreign pathogens. To avoid this, a number of pathogens have developed strategies to escape or limit the immune response allowing them to set up a more productive infection and in some cases persist in their hosts. Tumours present a more difficult problem as most of the genes they express are normal host genes and the selective pressure exerted on the immune system may be less than by microorganisms; tumours will die with their hosts and often develop after reproductive age. Like microorganisms tumours have developed a number of strategies to evade responses, which can generally be divided, into those preventing recognition and those which allow escape, or tolerance of a response.

FasL has an interesting pedigree in the field of tumour immunology. Although Fas is expressed on a number of non-lymphoid tissues, such as the liver, FasL expression is tightly regulated, being found predominantly on activated lymphocytes. The role of FasL in the immune system is well studied; it is one of the mechanisms used by lymphocytes to kill targets expressing Fas², and it has also been suggested that Fas can deliver an activating signal to T cells²² In addition, activated lymphocytes which co express Fas and FasL, become susceptible to apoptosis³. The importance of this for the control of peripheral T cell populations is illustrated by the lymphoproliferation and auto-immunity developed by humans or mice with mutations in Fas or FasL²³.

A few extra-lymphoid tissues including the brain, eye and testis have been shown to express FasL. Expression of FasL at such sites is proposed to limit inflammation and confer immunoprivilege by allowing them to kill infiltrating lymphocytes expressing Fas⁴⁻⁶. These observations incited much interest in several fields, most notably transplantation and tumour immunology. Initial observations demonstrated expression of FasL on some tumours where it was proposed to allow them to escape the immune response. Following these observations a number of investigators tested the effects in tumour cells or transgenic organs overexpressing FasL. Often the results were disappointing (from the tumours standpoint), tumour cells or transgenic tissues faired worse than their non-transfected counterparts⁹. The expression of FasL in a number of instances provoked an intense neutrophil infiltrate^{18, 24}. Neutrophils express Fas and can be activated by FasL to become cytotoxic²⁵. It is believed that this neutrophil activation leads to tumour rejection and blocking with soluble Fas-Fc fusion protein reduced neutrophil activation and killing of FasL transfected tumour cells²⁵. The inventors results using B16F10 transfected with a FasL mutant which fails to bind Fas confirm these results.

However it is not entirely clear why FasL transfected tumours induce such a brisk neutrophil infiltrate. Previous studies have suggested a role for IL1β as FasL transfected fibrosarcoma was not rejected in IL1β deficient mice²⁶. In addition soluble FasL can also attract neutrophils, although expression of a soluble form of FasL in tumour cells did not lead to their rejection, or to a neutrophil infiltrate^{11, 27, 28}. In this study the inventors tested rejection in mice deficient in MIP1α. These mice were also less efficient at rejecting the tumour suggesting a possible role for MIP1α as a neutrophil chemoattractant in FasL induced tumour rejection²⁹. Consistent with this finding is the recent demonstration that membrane-bound FasL leads to increase of mRNA expression of MIP1α and other proinflammtory mediators³⁰. As soluble FasL does not induce neutrophil infiltration the inventors examined the role of the cytoplasmic domain of FasL. This proline rich domain of around 70 amino acids is highly conserved between species and can bind the src kinase fyn in vitro¹⁴. Previous studies have shown a role for the FasL in the proliferation of CD8 cells and also cell cycle arrest of CD4 cells^{31, 32}. Thus it has been suggested that FasL may deliver a “reverse signal” via the cytoplasmic domain. In the inventors' experiments deletion of the cytoplasmic domain had no effect on the kinetics of tumour growth ex vivo (data not shown), and likewise had no effect on tumour rejection in vivo.

Although the initial rejection of FasL expressing tumour has been studied extensively little is known about how this affects long term tumour immunity. In a previous study a CDB dependent protective responses to lymphoma was induced by treatment with tumour transfected with FasL^{18, 19}. In the inventors' experiments depletion of CD8⁺ cells had no effect perhaps because B16F10 expresses low levels of MHC class I. Similarly depletion of CD4⁺ cells from protected animals did not reduce tumour rejection although transfer of CD4 cells and splenic B cells prevented tumour growth in 2/4 animals whilst slowing growth in the other 2/4 (data not shown). The B16F10 melanoma model has recently been shown to belong to the type of tumours that are weakly immunogenic because they grow as nodules “walled off” from the immune system and in addition it expresses low levels of MHC class I, preventing activation of anti-tumour immunity mediated by CD8⁺ lymphocytes³³. Thus the B16F10 is a particularly difficult tumour model in which to raise an adaptive immune response, and might be representative of many human tumours³⁴ that are clinically detectable.

To the inventors surprise serum provided almost complete protection against a secondary tumour challenge. The serum from protected mice can recognise surface determinants on B16F10 and the unrelated melanoma cell line K1735. It is impossible at this stage to say how broad the response is or which antigens on the melanoma are responsible for the protection afforded by serum transfer. However, the inventors have appreciated that their model may be used to screen for specific antibodies and/or specific tumour associated antigens which may be used as tumour vaccines. It is possible to produce monoclonal antibodies from the protected animals to define some of these antigens. However the inventors' screen of melanocyte differentiation antigens expressed in recombinant vaccinia virus has identified some antigens. gp100 and Trp1 responses were detected in the protected mice. Trp-1 can be expressed at the cell surface and passive transfer of antibodies against Trp-1 have been shown to induce rejection of melanoma in vivo^{35, 36}.

How FasL is able to elicit these responses is at present not entirely clear. It seems likely that the effect is multifactorial. Firstly the recruitment and activation of neutrophils by FasL is likely a crucial event as it not only allows early innate killing of the tumour, but also leads to the secretion of proinflammatory cytokines and further immune activation³⁷. Dendritic cells may be recruited by this milieu and induced to take up tumour cells/antigen. Furthermore FasL expressed on the tumour can help mature the DC's leading to increase expression of costimulatory molecules and consequent priming³⁸ of CD4 T cells which are clearly needed to establish this immune response (FIG. 2). The isotypes of the specific antibodies are IgG1, IgG2a and IgG2b indicating a mixed TH1/TH2 pattern. The use of other proinflammatory cytokines expressed on tumour cells such as GM-CSF, B7-1, MHC class II or cytokines may well work in a similar fashion by inducing effective DC maturation and cross priming39. Recent evidence suggests that the innate anti-tumour response mediated by natural killer cells modulates the development of the adaptive immune response is⁴⁰. Similarly it is possible that the innate immune response initiated by FasL induces the humoral anti-tumour response found in this study. The inventors' experiments suggest a role for ADCC in tumour destruction as the tumour rejection was much reduced in FcRγ^{-/-} mice.

Several monoclonal antibodies such as anti-CD20 and anti-HER2 are already used in the treatment of cancer and a larger number are currently undergoing clinical trials⁴¹. Antibodies are attractive for the treatment of cancer as they have many modes of action: blocking or stimulation of a cell surface receptor, cytotoxicity via complement or antibody dependent cell mediated cytotoxicity (ADCC). The inventors show herein that ADCC can account for some protection in this system. Finally, if inactive on these counts, conjugated antibody can be used to deliver drugs or toxins to cancer cells. Single monoclonals will have their limitations as they put a huge selective pressure on the tumour to lose antigen expression as do therapies inducing CD8⁺ T cells where loss of MHC class I will abolish responses against all antigens. In order to produce a truly effective therapeutic tumour vaccine/immunotherapy in humans it will probably be necessary to induce responses against multiple antigens using multiple effector mechanisms. The inventors' observations here may help in the definition of new cell surface tumour markers and may provide an additional route to stimulate a broader anti-tumour response.

Experimental Procedures

Tumour Cells

B16F10 were obtained from Professor I. Hart (London, UK) and from Prof. R. Zinkernagel (Zürich, Switzerland). Cultures of the melanoma cell line B16F10 were maintained in RPMI (Sigma) supplemented with 10% foetal calf serum (FCS), L-glutamine, penicillin-streptomycin. The K1735 is a melanoma cell line from C3H strain of mice and was obtained from Prof I. Fidler (University of Texas). MC57 is a methylcholanthrene-induced fibrosarcoma cell line from C57BL/6 strain of mice.

FasL was cloned into the pEGFP-C1 vector (Clontech) placing GFP at the amino terminus of FasL. Deletion mutants lacking the cytoplasmic chain of FasL (lacking 72 amino acids) and/or a single point mutation that abolishes binding to Fas (Y218R) were created by PCR and subsequently cloned into the same vector. B16F10, cells were transfected with the above constructs with DMRIE (Gibco) and selected on 1.5 mg/ml G418, single-cells were sorted by flow cytometry and cloned. Clones were screened for FasL expression using Nok-1 (Pharmingen) by FACS. The in vivo results are representative of several different clones. 5×10⁵ live or 1×10⁷ irradiated tumour cells were injected into C57BL/6 mice and tumour growth measured. C57BL/6 were bred in the inventors' own animal facility; the FcRγ^{-/-} were obtainedfrom Taconic (Germantown N.Y.).

Depletion/Transfer of CD4⁺ and CD8⁺ Lymphocytes

Pairs of hybridomas secreting anti-CD4 (YTS 191.1.2, YTS 3 3.1.2, both rat IgG2b) and anti-CD8 (YTS 169.4.2.1, YTS 156.7.7, both rat IgG2b) were used for depletions as described previously⁴². 100 μg of the pair of anti-CD4 or anti-CD8 antibodies injected intraperitoneally 3 and 1 day prior to injection of 5×10⁵ B16WT. One day after the last injection less than 1% of CD4⁺ or CD8⁺ were detected in peripheral blood by FACS.

Transfer of CD4⁺, CD8⁺, B Cells and Serum

CD4⁺ and CD8⁺ lymphocytes from spleen and inguinal lymph nodes were purified by positive selection using either directly conjugated beads to anti-CD4, anti-CD8 (Miltenyi Biotec) or using antibodies against. CD4 (Pharmingen), CD8 (Caltag) or B220 (Pharmingen) followed by anti-rat-beads. Purity of CD4, CD8 was between 70% for CD8⁺ and 95% for CD4⁺ cells. 10×10⁶ CD4 and 5×10⁶ CD8 per mouse were re-injected intravenously (i.v.) the day of purification. To obtain serum blood was clotted at 37° C. for 1 h, then kept at 4° C. for at least 1 h, spun at 20000 g for 10 min. 200 μl of serum per mouse was reinjected i.v. Purification of the Ig fraction of serum was performed with a protein L column (Pierce) according to the manufacturer's protocol. Protein L binds to the κ light chain and allows purification of IgM and IgG and possibly other murine antibody isotypes⁴³. 1.2 mg of the Ig fraction was re-injected per mouse. For all transfer experiments mice were challenged the day following transfer with 5×10⁵ B16WT.

Proliferation Assay with Dendritic Cells

Murine dendritic cells were isolated from bone marrow. Erythrocytes were depleted with lysis buffer (Flowgen). Cells were cultured with RPMI 5% FCS (Hyclone) with 500 U/ml IL-4 (Peprotech) and 1000 U/ml GM-CSF (Peprotech). Cultures were fed on day 2 and day 4 by aspirating off medium and adding fresh medium. In some experiments DCs were fed only on GM-CSF (200 U/ml), medium was changed on day 2 and 3 by discarding adherent and loosely adherent cells. Using this protocol, murine DCs have an immature phenotype on day 4. On day 4 soluble FasL-flag was added alone or crosslinked with 5 μg/ml of anti-flag. Alternatively DC's were incubated with irradiated (12000 rad) B16F10 stably transfected with FasL overnight. Allogeneic proliferation was set up on day 5 by culturing 10⁵ BALB/c spleen cells with 10⁴ C57BL/6 dendritic cells that have been matured previously in the presence or absence of FasL. Four days later proliferation was assayed 3H-thymidine incorporation.

Vaccinia Assay

Mice that were previously treated with B16FasL or not were infected intraperitoneally with 2×10⁶ PFU of vaccinia expressing different melanocyte antigens or control antigens. Both ovaries were harvested at indicated time points (5 days), and the vaccinia titres were determined on TK⁻ monolayers as described previously⁴⁴.

Flow Cytometry and Complement Lysis Assay

FACS staining with serum was performed at a concentration of 1/100, control serum was obtained from untreated or non-immunised mice with tumours. Second layer antibodies included anti-mouse Ig (Dako), anti-IgG1, IgG2a, IgG2b and IgG3. For the complement assay B16GF10 cells were labelled for 90 min with ⁵¹Cr, washed thoroughly and plated out at 2.5×10⁴ per well. Pooled serum from protected mice or normal

mouse serum was added at 1:5 dilution for 1 h, washed off, then rabbit complement (Low-Tox Cedarlane, Canada) was added at 1:10 dilution for 3 hours in a total volume of 80 μl. 35 μl was harvested and counted on a beta counter. Chromium release into the supernatant was determined by liquid scintillation.

TABLE 1
The primary B16FasL is rejected and induces tumour immunity

% mice tumour free

| | | secondary^{b} |
|---|---|---|
| Pretreatment/primary | primary^{a} | challenge |
| challenge (tumour type) | challenge | with B16WT |
| B16WT | 6% | N/A |
| 5 * 10⁵ live | (n = 34) | |
| B16FasL | 56% | 54% |
| 5 * 10⁵ live | (n = 123) | (n = 89) |
| B16WT | N/A | 10% |
| 10⁷ irradiated | | (n = 50) |
| B16FasL | N/A | 70% |
| 10⁷ irradiated | | (n = 90) |

^{a}Mice were either injected with 5 × 10⁵ B16FasL or 10⁷ irradiated B16FasL.
^{b}Four to eight weeks later tumour-free mice were challenged with 5 × 10⁵ B16F10 wild type tumour.

TABLE 2
CD8 lymphocytes do not confer protection
in B16FasL immunised mice

| | | Tumour free mice | |
|---|---|---|---|
| | | Experiment 1 | Experiment 2 |
| A | Isotype control depletion | 4/4 | 5/5 |
| | CD4+ depletion | 4/4 | 6/6 |
| | CD8⁺ depletion | 4/4 | 6/6 |
| B | Transfer of CD8⁺ | 0/3 | 0/4 |
| | Transfer of | 0/3 | ND |
| | CD4⁺ and CD8⁺ | | |
| | Transfer of | ND | 0/4 |
| | CD4⁺ depleted splenocytes | | |

A Protected mice were treated twice with a pair of αCD4 or αCD8 depleting antibodies, then challenged the following day with 5 × 10⁵ B16WT. Tumour growth was monitored over a period of 5 months.
B Lymphocytes were transferred from protected mice into naive recipients, then challenged with 5 × 10⁵ B16WT. Tumour growth was monitored over a period of 8 weeks.

References
1. Shankaran, V. et al. IFNgamma and lymphocytes prevent primary tumour development and shape tumour immunogenicity. Nature 410, 1107-1111. (2001).
2. Krammer, P. H. CD95's deadly mission in the immune system. Nature 407, 789-795. (2000).
3. Janssen, O., Sanzenbacher, R. & Kabelitz, D. Regulation of activation-induced cell death of mature T-lymphocyte populations. Cell Tissue Res 301, 85-99. (2000).
4. Bellgrau, D. et al. A role for CD95 ligand in preventing graft rejection. Nature 377, 630-632. (1995).
5. Griffith, T. S., Brunner, T., Fletcher, S. M., Green, D. R. & Ferguson, T. A. Fas ligand-induced apoptosis as a mechanism of immune privilege. Science 270, 1189-1192. (1995).
6. Saas, P. et al. Fas ligand expression by astrocytoma in vivo: maintaining immune privilege in the brain? J Clin Invest 99, 1173-1178. (1997).
7. O'Connell, J., Houston, A., Bennett, M. W., O'Sullivan, G. C. & Shanahan, F. Immune privilege or inflammation? Insights into the Fas ligand enigma. Nat Med 7, 271-274. (2001).
8. Walker, P. R., Saas, P. & Dietrich, P. Y. Tumor expression of Fas ligand (CD95L) and the consequences. Curr Opin Immunol 10, 564-572. (1998).
9. Green, D. R. & Ferguson, T. A. The role of Fas ligand in immune privilege. Nature Reviews Molecular Cell Biology 2, 917-924 (2001).
10. Schneider, P. et al. Characterization of Fas (Apo-1, CD95)-Fas ligand interaction. J Biol Chem 272, 18827-18833. (1997).
11. Hohlbaum, A. M., Moe, S. & Marshak-Rothstein, A. Opposing effects of transmembrane and soluble Fas ligand expression on inflammation and tumor cell survival. J Exp Med 191, 1209-1220. (2000).
12. Shimizu, M. et al. Fas/Apo-1 (CD95)-mediated apoptosis of neutrophils with Fas ligand (CD95L)-expressing tumors is crucial for induction of inflammation by neutrophilic polymorphonuclear leukocytes associated with antitumor immunity. Cell Immunol 207, 41-48. (2001).
13. Suda, T., Takahashi, T., Golstein, P. & Nagata, S. Molecular cloning and expression of the Fas ligand, a novel member of the tumor necrosis factor family. Cell 75, 1169-1178. (1993).
14. Hane, M., Lowin, B., Peitsch, M., Becker, K. & Tschopp, J. Interaction of peptides derived from the Fas ligand with the Fyn-SH3 domain. FEBS Lett 373, 265-268. (1995).
15. Alam, R., Kumar, D., Anderson-Walters, D. & Forsythe, P. A. Macrophage inflammatory protein-1 alpha and monocyte chemoattractant peptide-1 elicit immediate and late cutaneous reactions and activate murine mast cells in vivo. J Immunol 152, 1298-1303. (1994).
16. Lira, S. A. et al. Expression of the chemokine N51/KC in the thymus and epidermis of transgenic mice results in marked infiltration of a single class of inflammatory cells. J Exp Med 180, 2039-2048. (1994).
17. Dranoff, G. et al. Vaccination with irradiated tumor cells engineered to secrete murine granulocyte-macrophage colony-stimulating factor stimulates potent, specific, and long-lasting anti-tumor immunity. Proc Natl Acad Sci USA 90, 3539-3543. (1993).
18. Seino, K., Kayagaki, N., Okumura, K. & Yagita, H. Antitumor effect of locally produced CD95 ligand. Nat Med 3, 165-170. (1997).
19. Shimizu, M. et al. Induction of antitumor immunity with Fas/APO-1 ligand (CD95L)- transfected neuroblastoma neuro-2a cells. J Immunol 162, 7350-7357. (1999).
20. Townsend, A. et al. Defective presentation to class I-restricted cytotoxic T lymphocytes in vaccinia-infected cells is overcome by enhanced degradation of antigen. J Exp Med 168, 1211-1224. (1988).
21. Overwijk, W. W. et al. gp100 /pmel17 is a murine tumor rejection antigen: induction. of “self”- reactive, tumoricidal T cells using high-affinity, altered peptide ligand. J Exp Med 188, 277-286. (1998).
22. Kennedy, N. J., Kataoka, T., Tschopp, J. & Budd, R. C. Caspase activation is required for T cell proliferation. J Exp Med 190, 1891-1896. (1999).
23. Matiba, B., Mariani, S. M. & Krammer, P. H. The CD95 system and the death of a lymphocyte. Semin Immunol 9, 59-68. (1997).
24. Allison, J., Georgiou, H. M., Strasser, A. & Vaux, D. L. Transgenic expression of CD95 ligand on islet beta cells induces a granulocytic infiltration but does not confer immune privilege upon islet allografts. Proc Natl Acad Sci USA 94, 3943-3947. (1997).
25. Chen, J. J., Sun, Y. & Nabel, G. J. Regulation of the proinflammatory effects of Fas ligand (CD95L). Science 282, 1714-1717. (1998).
26. Miwa, K. et al. Caspase 1-independent IL-1beta release and inflammation induced by the apoptosis inducer Fas ligand. Nat Med 4, 1287-1292. (1998).
27. Seino, K. et al. Chemotactic activity of soluble Fas ligand against phagocytes. J Immunol 161, 4484-4488. (1998).
28. Ottonello, L., Tortolina, G., Amelotti, M. & Dallegri, F. Soluble Fas ligand is chemotactic for human neutrophilic polymorphonuclear leukocytes. J Immunol 162, 3601-3606. (1999).
29. Lee, S. C. et al. Cutaneous injection of human subjects with macrophage inflammatory protein-1 alpha induces significant recruitment of neutrophils and monocytes. J Immunol 164, 3392-3401. (2000).
30. Hohlbaum, A. M., Gregory, M. S., Ju, S. T. & Marshak-Rothstein, A. Fas ligand engagement of resident peritoneal macrophages in vivo induces apoptosis and the production of neutrophil chemotactic factors. J Immunol 167, 6217-6224. (2001).
31. Desbarats, J., Duke, R. C. & Newell, M. K. Newly discovered role for Fas ligand in the cell-cycle arrest of CD4+ T cells. Nat Med 4, 1377-1382. (1998).
32. Suzuki, I. & Fink, P. J. Maximal proliferation of cytotoxic T lymphocytes requires reverse signaling through Fas ligand. J Exp Med 187, 123-128. (1998).
33. Ochsenbein, A. F. et al. Roles of tumour localization, second signals and cross priming in cytotoxic T-cell induction. Nature 411, 1058-1064. (2001).
34. Pardoll, D. T cells and tumours. Nature 411, 1010-1012. (2001).
35. Hara, I., Takechi, Y. & Houghton, A. N. Implicating a role for immune recognition of self in tumor rejection: passive immunization against the brown locus protein. J Exp Med 182, 1609-1614. (1995).
36. Naftzger, C. et al. Immune response to a differentiation antigen induced by altered antigen: a study of tumor rejection and autoimmunity. Proc Natl Acad Sci USA 93, 14809-14814. (1996).
37. Fuchs, E. J. & Matzinger, P. Is cancer dangerous to the immune system? Semin Immunol 8, 271-280. (1996).
38. Rescigno, M. et al. Fas engagement induces the maturation of dendritic cells (DCs), the release of interleukin (IL)-1beta, and the production of interferon gamma in the absence of IL-12 during DC-T cell cognate interaction: a new role for Fas ligand in inflammatory responses. J Exp Med 192, 1661-1668. (2000).
39. Smyth, M. J., Godfrey, D. I. & Trapani, J. A. A fresh look at tumor immunosurveillance and immunotherapy. Nat Immunol 2, 293-299. (2001).
40. Kelly, J. M. et al. Induction of tumor-specific T cell memory by NK cell-mediated tumor rejection. Nat Immunol 3, 83-90. (2002).
41. Houghton, A. N. & Scheinberg, D. A. Monoclonal antibody therapies-a ‘constant’ threat to cancer. Nat Med 6, 373-374 (2000).
42. Qin, S., Cobbold, S., Tighe, H., Benjamin, R. & Waldmann, H. CD4 monoclonal antibody pairs for immunosuppression and tolerance induction. Eur J Immunol 17, 1159-1165. (1987).
43. Svensson, H. G., Hoogenboom, H. R. & Sjobring, U. Protein LA, a novel hybrid protein with unique single-chain Fv antibody- and Fab-binding properties. Eur J Biochem 258, 890-896. (1998).
44. Binder, D. & Kundig, T. M. Antiviral protection by CD8+ versus CD4+ T cells. CD8+ T cells correlating with cytotoxic activity in vitro are more efficient in antivaccinia virus protection than CD4-dependent IL. J Immunol 146, 4301-4307. (1991).

1. An immunocomplex for raising an immune response in an individual against an antigenf said immunocomplex comprising the antigen and Fas ligand (FasL) as an adjuvant.

2. An immunocomplex according to claim 1 wherein the immune response is a CD4 immune response.

3. An immunocomplex according to claim 1 wherein the antigen is a tumour associated antigen.

4. A immunocomplex according to claim 1 wherein the antigen is a bacterial or viral antigen.

5. An immunocomplex according to claim 1 wherein said immunocomplex is a fusion protein of the antigen and FasL.

6. An immunocomplex according to claim 1 wherein said immunocomplex is a tumour cell expressing FasL.

7. An immunocomplex according to claim 6 wherein the tumour cell is a melanoma cell.

8. An immunocomplex according to claim 1 further comprising an agent capable of complementing the FasL adjuvant.

9. An immunocomplex according to claim 8 wherein the agent is capable of depleting CD25 expressing cells in vivo.

10. An immunocomplex according to claim 8 wherein the agent is an anti-CD25 monoclonal antibody.

11. A nucleic acid molecule encoding an immunocomplex according to claim 5.

12. A pharmaceutical composition comprising an immunocomplex claim 1.

13. A method of providing an immunocomplex comprising a tumour associated antigen and FasL, said method comprising transfecting a tumour cell with FasL such that the ligand is expressed by the transfected cell along with the tumour associated antigens.

14. A method of providing an immunocomplex comprising a pathogen associated antigen and FasL, said method comprising transfecting or transform ng a pathogen cell w th FasL such that the ligand is expressed by the transfected cell along with the pathogen associated antigens.

15. (canceled)

16. A method of inducing an immune response in an individual against an antigen, comprising the step of administering to said individual an immunocomplex according to claim 1.

17. A method of providing an immunocomplex for inducing an immune response in an individual against a tumour cell, said method comprising obtaining a tumour cell from said individual; and transfecting said cell with FasL such that said cell expresses FasL.

18. (canceled)

19. A method of treating cancer in an affected individual, comprising the step of administering a transfected tumour cell produced by a method according to claim 17.

20. A method of identifying cell-specific antibodies comprising (a) transfecting a cell with FasL; (b) vaccinating a test animal with said transfected cell; (c) collecting serum from said test animal; and (d) identifying antibodies specific for said cell from the serum.

21. A method according to claim 20 further comprising the step (e) producing monoclonal antibodies from said identified antibodies.

22. A method according to claim 20 further comprising the step of producing a pharmaceutical composition comprising said antibodies.

23. A method of identifying specific cell associated antigens comprising (a) contacting an antibody identified by a method according to claim 20 with a plurality of potential cell associated antigens; (b) identifying specific binding between said antibody and an antigen; and (c) characterising said antigen.

24. A method according to claim 23 wherein said potential cell associated antigens are derived from the cell used to identify the tumour specific antibodies.

25. A method according to claim 23 wherein the potential cell associated antigens are displayed using an expression library or fixed to a solid support.

26. A method according to claim 23 wherein the specific binding between said antibody and an antigen is identified by a labelling technique.

27. A method according to claim 20 wherein said cell is a tumour cell, and said cell associated antigens are tumour associated antigens.

28. A method of inducing an immune response in an individual against an antigen, comprising the step of administering to said individual an immunocomplex as produced by the method of claim 13.

29. A method of inducing an immune response in an individual against an antigen, comprising the step of administering to said individual a nucleic acid according to claim 11.

30. A method of inducing an immune response in an individual against an antigen, comprising the step of administering to said individual pharmaceutical composition according to claim 12.

31. A pharmaceutical composition comprising a nucleic acid according to claim 11.

32. A method of inducing an immune response in an individual against an antigen, comprising the step of administering to said individual pharmaceutical composition according to claim 31.
